(19) United States
(12) Patent Application Publication
(10) Pub. No.: US 2020/0000318 A1
(43) Pub. Date: Jan. 2, 2020

(54) CABLE ACTUATION MECHANISM FOR STEERABLE ENDOSCOPE

(71) Applicant: ELECTRONICS AND TELECOMMUNICATIONS RESEARCH INSTITUTE, Daejeon (KR)

(72) Inventors: Jung Wook SUH, Daegu (KR); Soo In LEE, Daejeon (KR); Eun Chang CHOI, Daegu (KR)

(21) Appl. No.: 16/452,200

(22) Filed: Jun. 25, 2019

(30) Foreign Application Priority Data
Jun. 29, 2018 (KR) 10-2018-0075377
Jan. 25, 2019 (KR) 10-2019-0010196

(51) Int. Cl. A61B 1/005

(52) U.S. Cl. CPC A61B 1/0057; A61B 1/0055

(57) ABSTRACT

The present disclosure relates to a cable actuation mechanism configured to stably actuate a bending joint part located at an end of an endoscope, the cable actuation mechanism provided in the present disclosure has a hinge joint structure which may form a mechanically complete match with the bending joint part at the end including a plurality of rolling joints, and a plurality of pulleys are applied for smooth operation of a cable. The present disclosure also relates to an endoscope cable actuation mechanism configured to cause an occurrence of motion of one degree of freedom or two degrees of freedom or more according to a structure of the targeted bending joint part and applicable to a conventional manual-type endoscope and also to a driving part of a surgical robot.

### CROSS-REFERENCE TO RELATED APPLICATION

This application claims priority to and the benefit of Korean Patent Application Nos. 2018-0075377 and 2019-0010196, filed on Jun. 29, 2018 and Jan. 25, 2019, the disclosure of which is incorporated herein by reference in its entirety.

### BACKGROUND

### 1. Field of the Invention

The present disclosure relates to a cable actuation mechanism configured to stably actuate a target such as a working tool located at an end of an endoscope or the like using a bending joint part.

### 2. Discussion of Related Art

Each of mechanisms including a steerable endoscope and a laparoscopic surgical tool has a small joint at an end thereof and is generally connected to a steering lever of a driving part such as a hand grip part or the like configured to steer the mechanisms by a long and flexible wire cable or rope.

The cable serves as a transmission which transfers motions of hands of a user (such as a doctor, a nurse, or the like) to the joint at the end of the mechanism. The above is highly similar to a case in which motions from a muscle of a human body are transferred to a joint through the tendon. Accordingly, actuation of the joint by the cable is also referred to as tendon-actuation.

Since a wire cable can transfer only a pulling force, a pair of cables are Fnecessary to move one joint in both rightward and leftward directions.

Like an 8 mm tool used in a da Vinci surgical robot, generally, when a joint part pulley on which a cable transmission is wound has a circular shape, a driving spool having a circular shape the same as the above may be used in a driving part (i.e., a driver).

However, if a shape of the joint part pulley on which the cable is wound has a non-circular shape or a corresponding structure for miniaturization of the joint or the like, a conventional circular-shaped spool driver may increase inaccuracy of motion due to cable slack.

FIGS. 1A to 1E illustrate a cable driving method for a non-circular joint pulley 13, wherein FIG. 1A illustrates a case in which cable slack 15 occurs due to use of a conventional circular driving spool 11; FIGS. 1B to 1D illustrate a method of applying an elastic body 17 to prevent the cable slack; and FIG. 1E illustrates a method of using a plurality of actuators (driving spools). As shown in FIGS. 1B to 1D, although a problem of the cable slack can be somewhat solved by adding the elastic body 17, a large hysteresis occurs when an external force is changed. Further, in another method, as shown in FIG. 1E, two or more actuators (i.e., driving spools or briefly, spools) can be used to drive a single joint, but the method not only causes an increase of the volume and cost of a driving part but also is difficult to apply to a manual instrument not employing a motor.

FIGS. 2A to 2C conceptually illustrate a case in which a non-circular driver (the driving spool) is used to drive the non-circular joint pulley, in which FIG. 2A illustrates application of a driving spool having a shape which is the same as that of the pulley, FIG. 2B illustrates application of a driving spool having a shape which is not the same as that of the pulley but which is valid, and FIG. 2C illustrates a mechanism for providing pre-tension to the cable. This is harmoniously pulling and releasing a pair of driving cables in a mechanical way to match the structure of the joint. However, since the identical or valid shaped joint pulley and the driving spool can be adapted in a limited case, a design of the driver according to a shape of the joint should be appropriately performed.

Further, FIGS. 3A to 3D illustrate a shape of a cable driving joint, wherein FIG. 3A illustrates a general joint structure using a circular driving pulley, like a 5 mm tool of the da Vinci, FIG. 3B illustrates a hinge structure in which a pulley is removed to reduce the joint (that is, a pulleyless hinge joint), FIG. 3C illustrates a compliant or continuum joint driven using own elasticity of a structure, and FIG. 3D illustrates a pulleyless rolling joint miniaturized through rolling contact between links.

FIGS. 4A to 4C illustrates a bending joint part structure applied to an endoscope joint, wherein FIG. 4A illustrates a bending joint part configured to connect a plurality of pulleyless hinge joints, FIG. 4B illustrates a continuum bending joint, and FIG. 4C illustrates a bending joint part including a plurality of pulleyless rolling joints.

In the above, in FIGS. 3A to 3D illustrating a structure of a remotely driven joint driven by the cable, since the conventional shape in FIG. 3A is stable but has a limit for miniaturization, and other structures in FIGS. 3B and 3C can be miniaturized but each can cause friction and abrasion due to a scratch between a driving cable and the structure, the bending joint part is generally being configured and used to secure a broad actuation area as shown in FIGS. 4A to 4C.

Since cable tension in the joint of the structures in a cable of FIGS. 3A and 3B is difficult to maintain, a bending joint part having a structure like FIG. 3C can be used. Fortunately, since a joint having a pulleyless rolling joint shape like FIGS. 3D and 4C can be appropriately matched with a driving part of a pulleyless hinge joint structure (see FIG. 3B), a cable driver can be designed using the above.

### SUMMARY OF THE INVENTION

The present disclosure is directed to providing a cable actuation mechanism capable of stably driving a target located at an end of an endoscope using a bending joint part.

According to an aspect of the present disclosure, there is provided an endoscope cable actuation mechanism including a bending joint part connected to a target; a driving part configured to drive the bending joint part; and at least one pair of wire cables configured to connect the bending joint part and the driving part, wherein the bending joint part comprises at least one joint among a pulleyless rolling joint, a pulleyless hinge joint, and a continuum joint, and the driving part comprises a driving main body; the pulleyless rolling joint or the pulleyless hinge joint coupled to the driving main body; and a driving bar connected to the pulleyless rolling joint or the pulleyless hinge joint of the driving main body and directly or indirectly connected to the wire cables.

The endoscope cable actuation mechanism may further comprise a plurality of idlers disposed at both sides of the driving main body so that the wire cables come into contact with the plurality of idlers and are bent and configured to support and guide movement of the wire cables between the driving main body and the driving bar.

The endoscope cable actuation mechanism may further comprise first sub idlers located at an edge of an inner wall of the driving main body and disposed in pairs at both sides of the driving main body so that the wire cables curvedly come into contact with and pass between the pair of first sub idlers.

The endoscope cable actuation mechanism may further comprise a pair of second sub idlers located in pairs at both sides of the driving bar and configured to guide movement of the wire cables.

Multiple pairs of wire cables may connect the bending joint part and the driving part for at least two degrees of freedom of the target.

The bending joint part may form a yawing motion and a pitching motion with respect to the driving part by the multiple pairs of wire cables and may form a motion in which the yawing motion and the pitching motion are complexly applied.

The at least one joint among the pulleyless rolling joint, the pulleyless hinge joint, and the continuum joint may be used as a joint for each of the yawing motion and the pitching motion, and the joints of the yawing motion and the pitching motion may be alternately disposed in the bending joint part, and the driving part may comprise a yaw driver and a pitch driver for the yawing motion and the pitching motion.

According to another aspect of the present disclosure, there is provided an endoscope cable actuation mechanism including a bending joint part connected to a target; a driving part configured to drive the bending joint part; and at least one pair of wire cables configured to connect the bending joint part and the driving part, wherein the bending joint part comprises at least one joint among a pulleyless rolling joint, a pulleyless hinge joint, and a continuum joint, and the driving part comprises a driving main body; the pulleyless rolling joint or the pulleyless hinge joint coupled to the driving main body; and a driving bar connected to the pulleyless rolling joint or the pulleyless hinge joint of the driving main body and directly or indirectly connected to the wire cables, the endoscope cable actuation mechanism further including a plurality of idlers disposed at both sides of each of the driving main body and the driving bar so that the wire cables come into contact with the plurality of idlers and are bent and configured to support and guide movement of the wire cables between the driving main body and the driving bar, wherein the plurality of idlers comprise first idlers located at both sides of the driving main body; second idlers located at both sides of the driving main body to be close to the first idlers; and third idlers located at both sides of the driving bar, and each of the wire cables has one end portion which surrounds the first idler and is grounded and fixed to the driving main body and further comprises a wire loop cable having a loop shape and configured to surround and connect the second idler and the third idler.

Multiple pairs of wire cables may connect the bending joint part and the driving part for at least two degrees of freedom of the target.

The bending joint part may form a yawing motion and a pitching motion with respect to the driving part by the multiple pairs of wire cables and may form a motion in which the yawing motion and the pitching motion are complexly applied.

The at least one joint among the pulleyless rolling joint, the pulleyless hinge joint, and the continuum joint may be used as a joint for each of the yawing motion and the pitching motion, and the joints of the yawing motion and the pitching motion may be alternately disposed in the bending joint part, and the driving part may comprise a yaw driver and a pitch driver for the yawing motion and the pitching motion.

The wire cables may be disposed to connect the yaw driver and the pitch driver, and the driving part may have one end portion of the wire cable which is wound therearound and connected to be coupled to the driving main body, and may further comprise a fixing spool to which the plurality of wire cables forming different motions are fixed.

According to still another aspect of the present disclosure, there is provided an endoscope cable actuation mechanism including a bending joint part connected to a target; a driving part configured to drive the bending joint part; and at least one pair of wire cables configured to connect the bending joint part and the driving part, wherein the bending joint part comprises at least one joint among a pulleyless rolling joint, a pulleyless hinge joint, and a continuum joint, and the driving part comprises a driving main body; the pulleyless rolling joint or the pulleyless hinge joint coupled to the driving main body; and a driving bar connected to the pulleyless rolling joint or the pulleyless hinge joint of the driving main body and directly or indirectly connected to the wire cables, the endoscope cable actuation mechanism further including a plurality of idlers disposed at both sides of each of the driving main body and the driving bar so that the wire cables come into contact with the plurality of idlers and are bent and configured to support and guide movement of the wire cables between the driving main body and the driving bar, wherein the plurality of idlers comprise first idlers located at an inlet of the driving main body from the bending joint part; second idlers located to be spaced apart from the first idlers; third idlers located to be spaced apart from the second idlers; and fourth idlers located at the driving bar, the driving part is classified into a yaw driver and a pitch driver for a yawing motion and a pitching motion, each of the yaw driver and the pitch driver has the first to fourth idlers located at both sides thereof, and the wire cables sequentially come into contact with the first idler, the second idler, the third idler, and the fourth idler and then with the third idler and the second idler of one of the yaw driver and the pitch driver, and sequentially come into contact with the first idler, the second idler, the third idler, and the fourth idler and then with the third idler and the second idler of the other one of the yaw driver and the pitch driver to have one ends fixed to the driving main body.

Multiple pairs of wire cables may connect the bending joint part and the driving part for at least two degrees of freedom of the target.

The bending joint part may form the yawing motion and the pitching motion with respect to the driving part by the multiple pairs of wire cables and may form a motion in which the yawing motion and the pitching motion are complexly applied.

The at least one joint among the pulleyless rolling joint, the pulleyless hinge joint, and the continuum joint may be used as a joint for each of the yawing motion and the pitching motion, and the joints of the yawing motion and the pitching motion may be alternately disposed.

The wire cables may be disposed to connect the yaw driver and the pitch driver, and the driving part may have one end portion of the wire cable which is wound therearound and connected to be coupled to the driving main body, and may further comprise a fixing spool to which the plurality of wire cables forming different motions are fixed.

Fixing spools may be located and disposed in pairs at both sides of each of the yaw driver and the pitch driver and a plurality of wire cables forming different motions may be wound around and connected to the fixing spools.

The wire cables may be disposed at predetermined angle intervals along a circumferential direction and have freedom degrees of motion with respect to an axis of the bending joint part according to the number of the pairs of wire cables.

The driving part may further comprise sliding idlers located to be close to the bending joint part from the first idlers and configured to adjust tension of the wire cables while sliding according to the tension of the wire cables as the wire cables are disposed so as to surround.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIGS. 1A to 1E are views illustrating cable driving methods for a non-circular joint;

FIGS. 2A to 2C are views illustrating cable driving methods of a non-circular joint using a non-circular driving spool;

FIGS. 3A to 3D are views illustrating a shape of a cable driving joint;

FIGS. 4A to 4C are views illustrating a structure of a bending joint part applied to an endoscopic joint;

FIG. 5 is a conceptual diagram of cable driving for driving a one-degree-of-freedom bending joint part including a plurality of pulleyless rolling joints in an endoscope cable actuation mechanism according to an embodiment of the present disclosure;

FIG. 6 is a conceptual diagram in which miniaturized idlers are additionally applied to prevent a scratch of a cable at an edge of components of a driving part in an endoscope cable actuation mechanism according to another embodiment of the present disclosure;

FIG. 7 is a conceptual diagram of cable driving using a cable loop wound around a plurality of duplicated idlers in an endoscope cable actuation mechanism according to still another embodiment of the present disclosure;

FIGS. 8A to 8E are conceptual diagrams of cable driving for a two-degree-of-freedom joint of yawing and pitching in an endoscope cable actuation mechanism according to yet another embodiment of the present disclosure;

FIGS. 9A to 9E are conceptual diagrams of modified cable driving in which sliding idlers which may perform translation movement are applied in an endoscope cable actuation mechanism according to yet another embodiment of the present disclosure;

FIG. 10 is a layout diagram of twelve cables configured to drive the two-degree-of-freedom joint in an endoscope cable actuation mechanism according to yet another embodiment of the present disclosure;

FIG. 11 is a three-dimensional detailed design view of the two-degree-of-freedom joint and a cable driving part configured to drive the two-degree-of-freedom joint in an endoscope cable actuation mechanism according to yet another embodiment of the present disclosure; and

FIG. 12 illustrates a joint bending state view in various directions of a bending joint part according to cable driving by steering of a steering lever in an endoscope cable actuation mechanism according to yet another embodiment of the present disclosure.

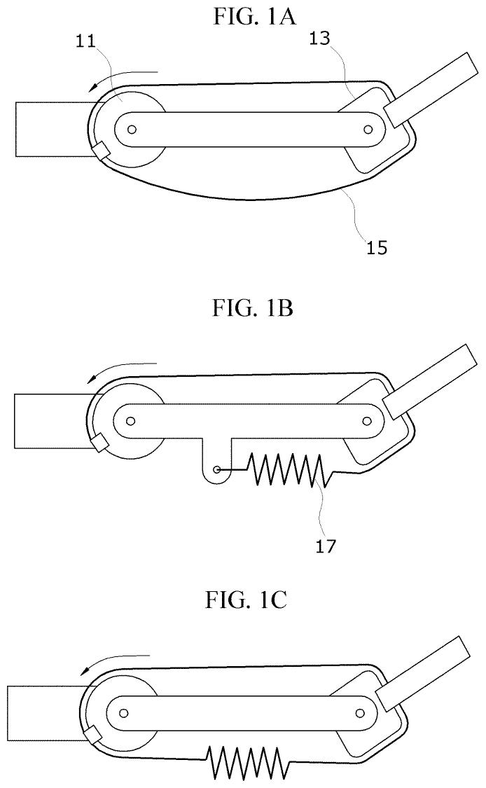

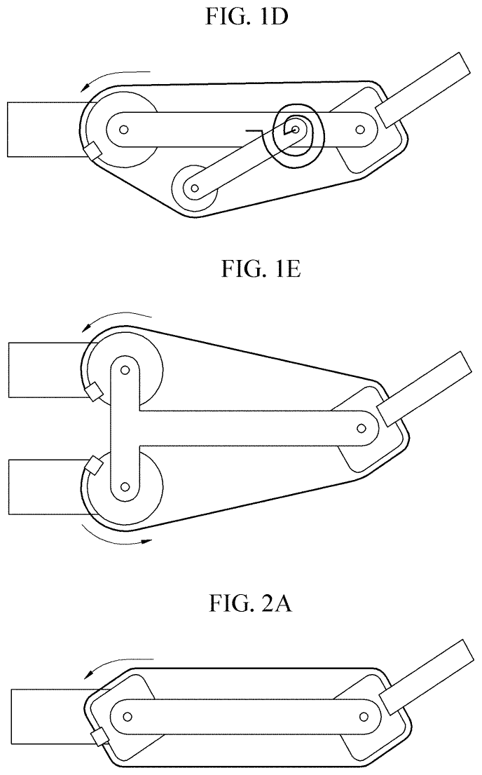

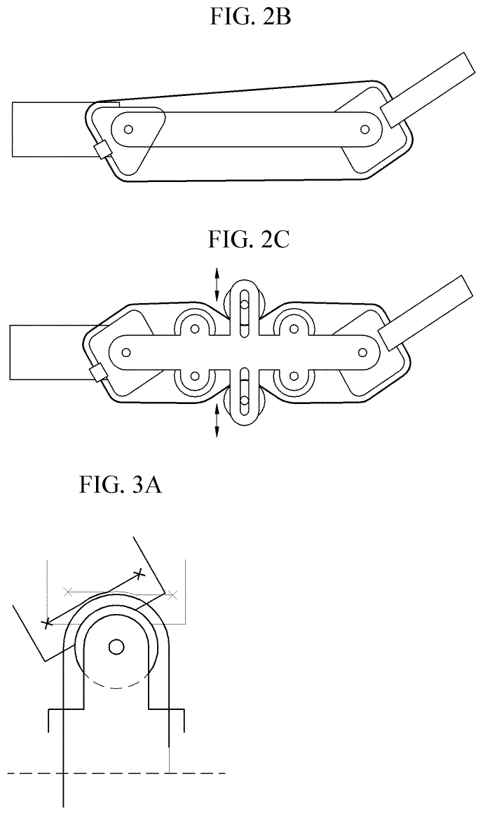

### DETAILED DESCRIPTION OF EXEMPLARY EMBODIMENTS

Hereinafter, various embodiments of the present disclosure are described by particular embodiments shown in the accompanying drawings. Differences between the embodiments are not exclusive items and should be complexly understood by the drawings, and particular shapes, structures and characteristics disclosed according to the embodiments without departing from the technical spirit and the scope of the present disclosure may be implemented as other embodiments.

Locations or disposition of separate elements according to embodiments of the present disclosure may be changed and should be understood as a combination of the drawings, similar reference numerals in the drawings may refer to the same or similar functions through various aspects, and a length, an area, a thickness, and the like and specific shapes thereof may be exaggerated for convenience of a description.

A direction and a location in the drawing is described according to an XYZ orthogonal coordinates system, and a frontward and backward direction or a vertical and lateral direction determines the direction and location according to an XY coordinate planometer and a YZ coordinate planometer. A unit, a module, a part, a member, or an arbitrary structure in which child elements are not described is assumed to include or be capable of including general child elements to have a granted function and is not limited to child elements shown in the drawings or a detailed structure. Elements which are shown but of which descriptions thereof are omitted because the descriptions are general contents should be understood to be included in detailed descriptions of the embodiments.

Terms which are used should be understood to have characteristics corresponding to means in a dictionary of general Chinese characters, Korean characters, or English characters or terms used in a corresponding field except particularly defined terms. When one element is mentioned to be “included”, “configured”, or “provided”, other elements may be further included. A case in which the element is fixed, bound, coupled, or connected refers to a case in which movement and a motion is completely or partially limited. On the other hand, rotation and hinge refers to a case in which an object may partially or completely rotate to move.

FIG. 5 is a conceptual diagram of cable driving for a one-degree-of-freedom bending joint part 100 including a plurality of pulleyless rolling joints 111 in an endoscope cable actuation mechanism according to an embodiment of the present disclosure.

Referring to FIG. 5, the endoscope cable actuation mechanism according to the embodiment of the present disclosure includes a bending joint part 100 connected to a target such as a working tool, a driving part 130 configured to drive the bending joint part 100, and at least one pair of wire cables 120 configured to connect the bending joint part 100 and the driving part 130.

The bending joint part 100 includes pulleyless rolling joints 111. The pulleyless rolling joints 111 allow curving of a joint due to three convex curved surface parts which come into contact with each other serially. The pulleyless rolling joints 111 may be curved by a tensile force of the wire cables 120 which pass through both sides thereof. In FIG. 5, one unit joint among unit joints forming the bending joint part 100 is shown.

The driving part 130 includes a driving main body 131, a pulleyless hinge joint 135 hinge-coupled to the driving main body 131, and a driving bar 136 connected to the pulleyless hinge joint 135 of the driving main body 131 and directly connected to the wire cables 120. The driving bar 136 practically serves as a steering lever 180 configured to pull the wire cables 120.

Referring to FIG. 5, in the embodiment, a plurality of idlers disposed at both sides of the driving main body 131 may be further included, so that the wire cables 120 come into contact with the plurality of idlers and are bent. Also, the plurality of idlers are configured to support and guide movement of the wire cables 120 from the driving main body 131 to the driving bar 136. More specifically, routing idlers 141 configured to guide the wire cables 120 are located in the driving main body 131 between the bending joint part 100 and the driving bar 136 of the driving part 130. The routing idlers 141 guide the movement of the wire cables 120 by rolling and coming into contact with the wire cables 120 like pulleys or rollers.

The routing idlers 141 are disposed in pairs at both sides of the driving main body 131 along a longitudinal direction of the driving main body 131, and each of the wire cables 120 is bent and passes between the idlers of the pair of routing idlers 141. Practically, the pair of routing idlers 141 adjust the wire cable 120 at the bending joint part 100 so that the wire cable 120 corresponds to a connecting point of the driving bar 136 at the driving part 130.

FIG. 5 shows a basic concept of the cable driving provided by the present disclosure. A length for pulling one wire cable 120 and a length for releasing an opposite wire cable 120 do not have to be the same to drive the joint. For example, on the basis of the joint in FIG. 5, in the case in which the right wire cable 120 should be released by 10 mm when the left wire cable 120 is pulled by 8 mm, the cable driving part 130 may be configured using the pulleyless hinge joint 135 to completely satisfy such a relation between actuation amounts of the pair of wire cables 120.

In the embodiment, when critical design parameters of the plurality of pulleyless rolling joints 111 including one pulleyless rolling joint 111 or the bending joint part 100 which is operated as a unit thereof are particularly related to critical design parameters of the pulleyless hinge joint 135 configured to operate the plurality of pulleyless rolling joints 111, the two parts may be connected to each other to be used.

On the contrary to FIG. 5, the bending joint part 100 may include a structure of the pulleyless hinge joint 135 and the driving part 130 may include pulleyless rolling joints 111.

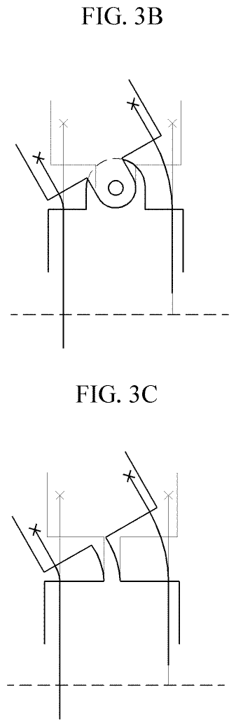

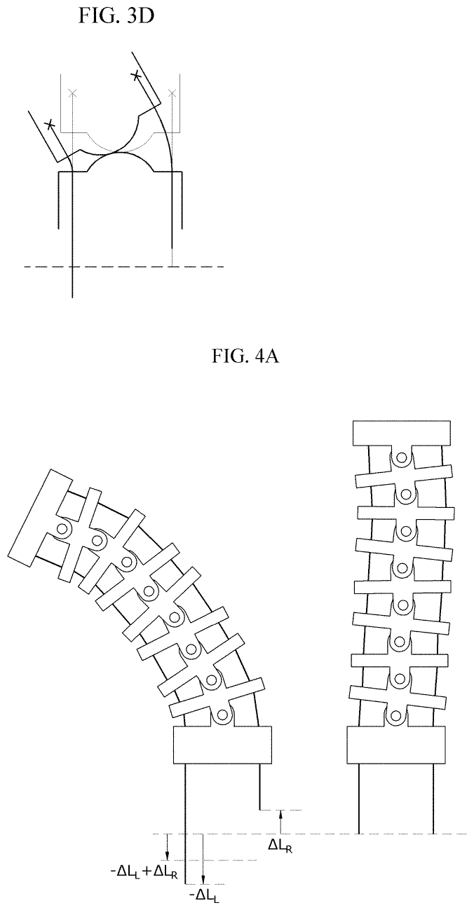

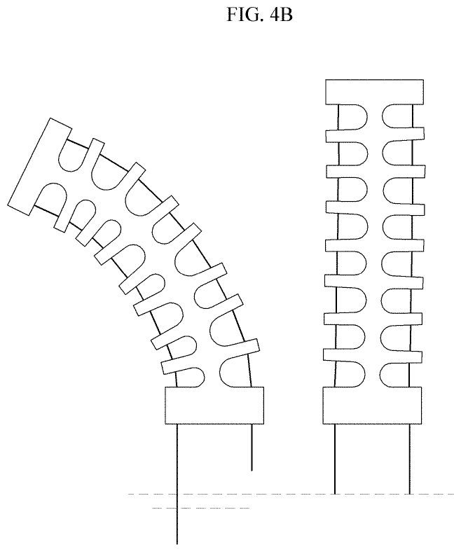

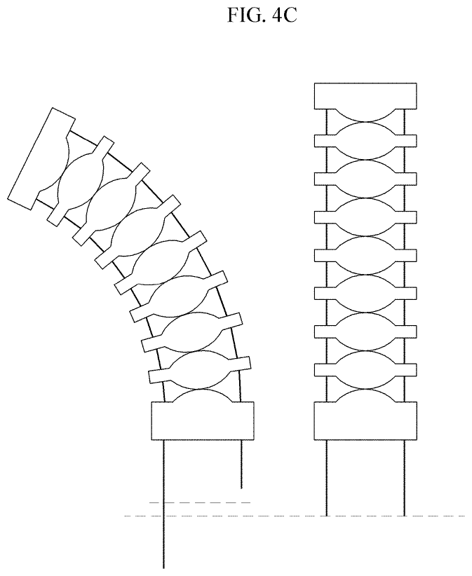

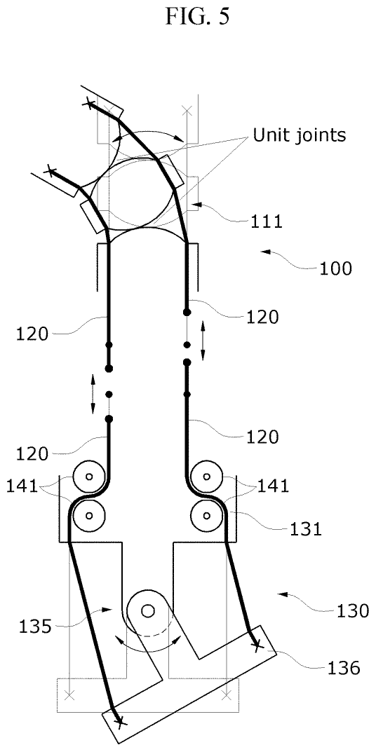

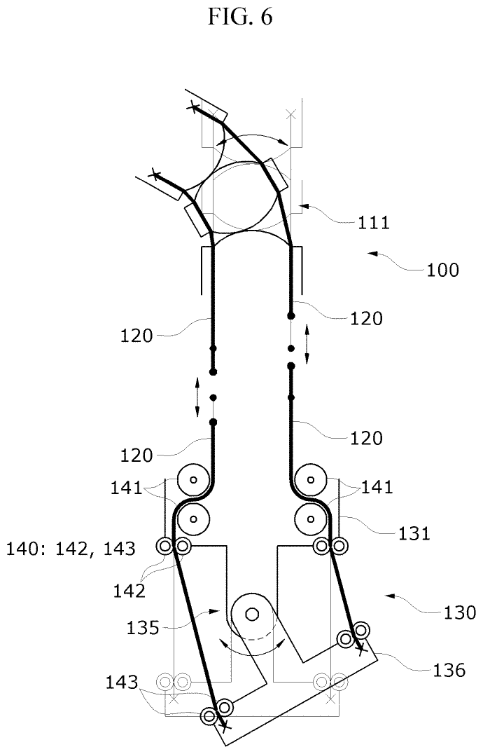

Further, although not shown, the bending joint part 100 includes a continuum joint shown in FIGS. 3A to 3D and 4A to 4C, and the pulleyless hinge joints 135 or the pulleyless rolling joints 111 may be applied to a connection part between the driving main body 131 of the driving part 130 and the driving bar 136.

FIG. 6 is a conceptual diagram in which miniaturized idlers are additionally employed to prevent cable scratch at edge portions of components of a driving part 130 in an endoscope cable actuation mechanism according to another embodiment of the present disclosure.

Referring to FIG. 6, the endoscope cable actuation mechanism, according to another embodiment of the present disclosure, may further include a plurality of idlers 140 disposed at both sides of a driving main body 131, so that wire cables 120 come into contact with the plurality of idlers 140 and are bent. The idlers 140, each of which has a small shaft diameter, are configured to support and guide movement of wire cables 120 from the driving main body 131 to a driving bar 136.

In this embodiment, the plurality of miniaturized idlers 140 may include: first sub idlers 142 located at an edge of an inner wall of the driving main body 131 and disposed in pairs at both sides of the driving main body 131 so that the wire cables 120 curvedly come into contact with and pass between the pair of first sub idlers 142; and second sub idlers 143 disposed in pairs at both sides of a driving bar 136 and configured to guide movement of the wire cables 120. The pair of first sub idlers 142 are disposed at the routing idlers 141 to guide the movement of the wire cables 120 to the driving bar 136. Further, the second sub idlers 143 guide and support the wire cables 120 to tangential points at the driving bar 136 from the first sub idlers 142 between the first sub idlers 142 and the driving bar 136 to restrict locations of one end portions of the wire cables 120 with respect to the tangential points.

Here, the first sub idlers 142 and the second sub idlers 143 may also be called as tangential idlers.

Since the first sub idlers 142 protect and guide the wire cables 120 at the edge of the driving main body 131, in the wire cable disposition structure shown in FIG. 5, an occurrence of friction due to a scratch between the driving joint and an edge of a component of the driving part 130 may be solved.

In the case of FIG. 6, since a length of the wire cable 120 may not be completely maintained, a variation width of tension of the wire cable 120 increases. In other words, probability of damage of the wire cable 120 increases because the tension applied to the wire cable 120 increases, or on the other hand, a slack phenomenon occurs in which the wire cable 120 becomes loose in a particular position because the tension decreases too much. The phenomenon may become severe when a diameter of the used idler increases.

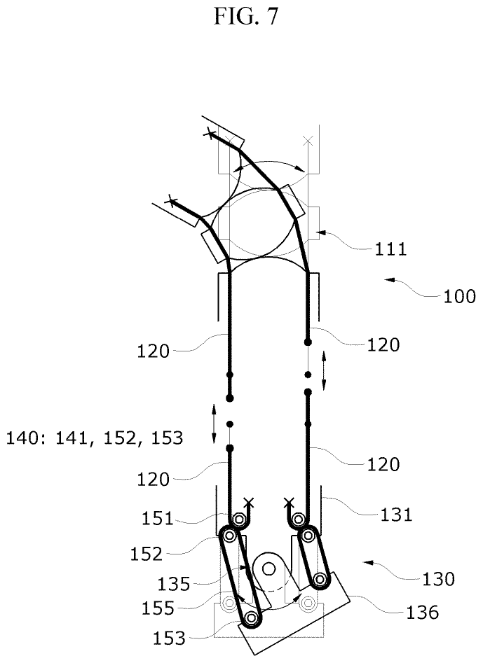

FIG. 7 is a conceptual diagram of cable driving using a cable loop wound around a plurality of duplicated idlers 140 in an endoscope cable actuation mechanism according to still another embodiment of the present disclosure.

Referring to FIG. 7, the plurality of duplicated idlers 140 include first idlers 151 located at both sides of a driving main body 131, second idlers 152 located at both sides of the driving main body 131 to be close to the first idlers 151, and third idlers 153 locate at both sides of a driving bar 136.

In each wire cable 120, one end portion surrounds the first idler 151 and is fixed to the driving main body 131. As an example, as described below, the wire cable 120 may be fixed to a fixing spool. In the embodiment, loop shaped wire loop cables 155 configured to surround and connect the second idlers 152 and the third idlers 153 are formed, and the loop cables 155 are the wire cables 120 disposed in a loop shape. Specifically, the wire cable 120 passes between the first idler 151 and the second idler 152, returns by surrounding the third idler 153 to come into contact with the second idler 152 again, passes between the first idler 151 and the second idler 152, and then is fixed to a tangential point of the driving main body 131. Accordingly, the wire loop cable 155 configured to connect the second idler 152 and the third idler 153 in the loop shape is formed. The wire loop cables 155 may be formed in an overlapping manner.

Accordingly, the embodiment is an embodiment to which driving of pulleyless rolling joints 111, in which one degree of freedom (DOF) is available, is applied. In the cable driving, since the first idlers 151 and the second idlers 152 which are duplicated are used, each of the wire cables 120 forms at least one wire loop cable 155.

The wire loop cable 155 forming the loop may decrease a size of a hinge joint for the driving, and the size decreases when more loops are wound. When ignoring the thickness of the idler, an ideal cable driving amount may be provided to the bending joint part 110 regardless of an angle of the hinge joint.

According to the embodiment, unlike a method of using tangential idlers which are the first and second sub idlers 142 and 143 of the embodiment shown in FIG. 6, since a length of the cable wound around the first to third idlers 151, 152, and 153 may be always uniformly maintained, the structure is not influenced by a radius of the idler. Since an influence due to the thickness of the idler is much smaller than the above, the influence is enough to be ignored. Accordingly, the driving part 130 of the wire cables 120 provided in the embodiment may stably maintain tension of the cable and drive the pulleyless rolling joints 111.

A plurality of pulleyless rolling joints 111 may be serially coupled to each other to configure a bending joint part 100 having a two degrees of freedom motion.

In the case in which a cable driver for tendon-actuation of the one-degree-of-freedom joint is expanded to two degrees of freedom, an actuation amount of cables important to be appropriately provided when motions of two joints driven in different directions are coupled.

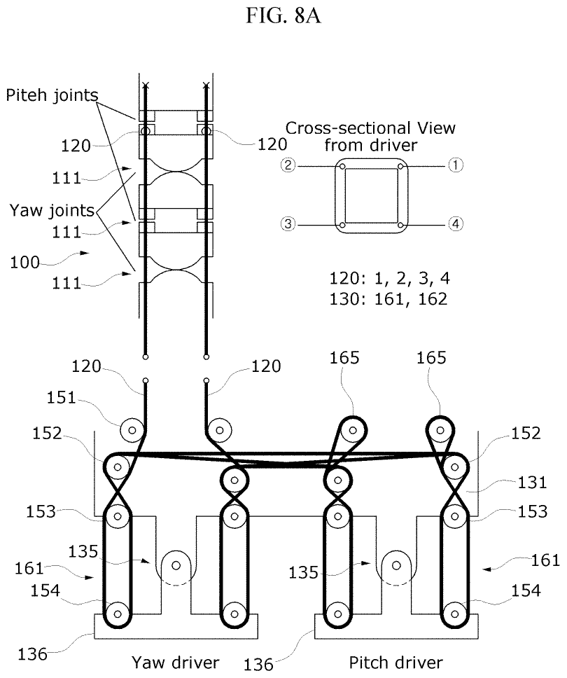

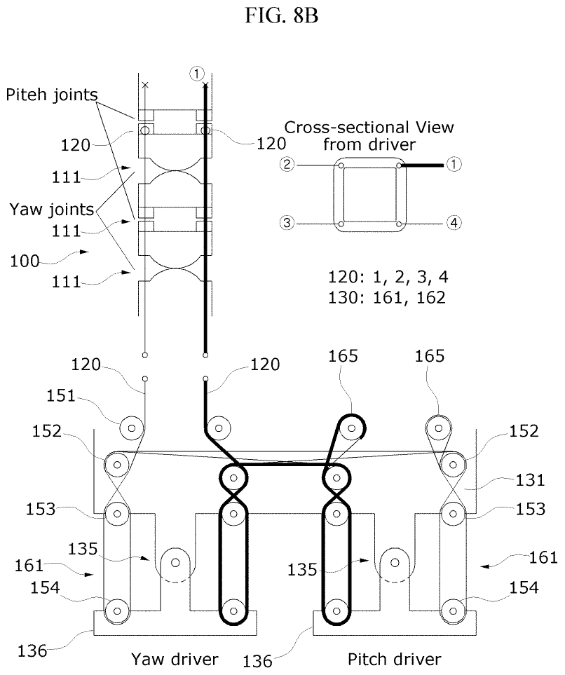

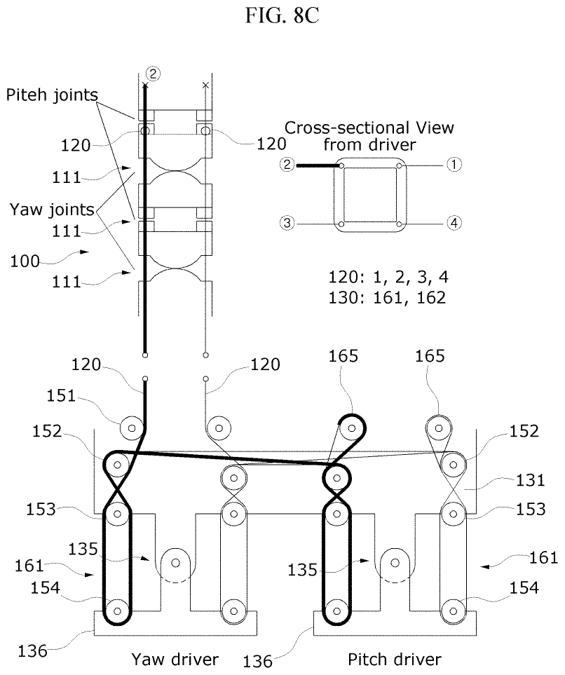

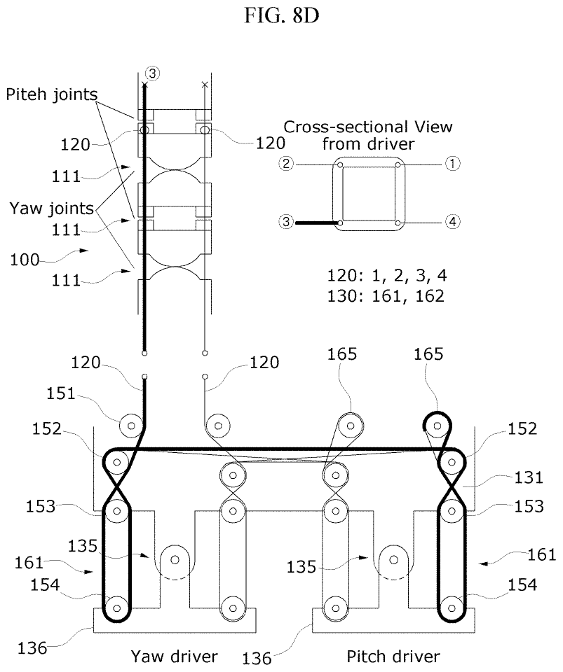

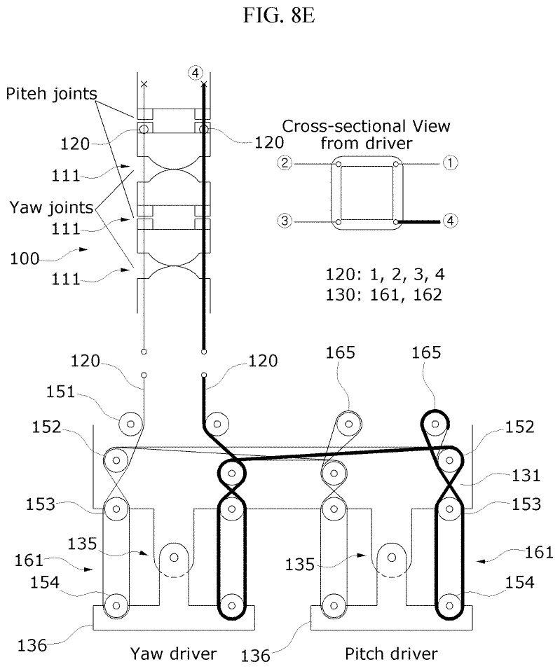

FIG. 8A is a conceptual diagram of cable driving for a two-degree-of-freedom joint including yawing and pitching in an endoscope cable actuation mechanism according to yet another embodiment of the present disclosure. FIGS. 8B to 8E separately show the relation between wire cables 120 and various idlers for easy understanding thereof.

Referring to FIG. 8A and FIGS. 8B to 8E, a bending joint part 100 according to yet another embodiment forms a yawing motion and a pitching motion with respect to a driving part 130 by multiple pairs of wire cables 120 and forms a motion in which the yawing motion and the pitching motion are complexly applied.

In the bending joint part 100, pulleyless rolling joints 111 are used as joints for the yawing motion and the pitching motion, and the joints of the yawing movement and the pitching movement are alternately disposed.

A driving part 130 is classified into a yaw driver 161 for the yawing motion and a pitch driver 162 for the pitching motion.

The wire cables 120 are disposed at a plurality of idlers to connect the yaw driver 161 and the pitch driver 162. One end portions of the wire cables 120 are wound around the plurality of idlers and finally fixed to the driving main body 131. Specifically, each of the plurality of idlers includes a first idler 151 located at an inlet of the driving main body 131 from the bending joint part 100, a second idler 152 located to be spaced apart from the first idler 151, a third idler 153 located to be spaced apart from the second idler 152, and a fourth idler 154 located at a driving bar 136.

In the yaw driver 161 and the pitch driver 162 of the endoscope cable actuation mechanism according to the embodiment, the second to fourth idlers 152 to 154 are disposed at both sides and form pairs. Further, fixing spools 165, to which the plurality of wire cables 120 forming different motions (see a cross-sectional view at an upper-right end in FIGS. 8A to 8E) are fixed, may be further included.

One end portion of one of the wire cables 120 sequentially comes into contact with the first idler 151, the second idler 152, the third idler 153, and the fourth idler 154 of the yaw driver 161, and then the third idler 153 and the second idler 152, and next, sequentially comes into contact with the second idler 152, the third idler 153, and the fourth idler 154 of the pitch driver 162, and then the third idler 153 and the second idler 152 to be disposed, and thus the one end portion is fixed to the fixing spool 165.

In the embodiment, the endoscope cable actuation mechanism may further include the fixing spools 165 to which the plurality of wire cables 120 forming the different motions are fixed.

As described above, the plurality of wire cables 120 forming the different motions are connected to the fixing spools 165, and motions such as yawing and pitching may be linked with each other to form other motions connected to a standard motion of the bending joint part 100 by the yaw driver 161 and the pitch driver 162.

A design of the cable driving part 130 for the one-degree-of-freedom pulleyless rolling joints 111 provided from the above may be applied to joints having at least two degrees of freedom.

Referring to FIG. 8A and FIGS. 8B to 8E, the embodiment shows a two-degree-of-freedom bending joint part 100 composed of a plurality of pulleyless rolling joints 111 including four yawing joint units and four pitching joint units alternately disposed and a conceptual design of a cable driver configured to drive the above. The cable driving part 130 includes two hinge joint structures provided with a plurality of idlers.

A left pulleyless hinge joint 135 causes a yawing operation (a lateral motion) of the bending joint part 100 and a right pulleyless hinge joint 135 causes a pitching operation (a vertical motion) of the bending joint part 100.

Although four cables for the motions in two directions are coupled to each other, the pitching operation and the yawing operation are completely decoupled from each other. In other words, the pulleyless rolling joints 111 of a yawing joint are not influenced even when the pulleyless rolling joints 111 corresponding to a pitching joint is moved by the pitch driver 162 at a right side, and the yawing operation by the pulleyless rolling joints 111 corresponding to the yawing joint is the same. However, when both the yaw driver 161 and the pitch driver 162 move together, the yawing operation and the pitching operation occur together.

On the basis of a disposition configuration of the above-described wire cables 120, the structure of the driving part 130 may be variously modified and designed.

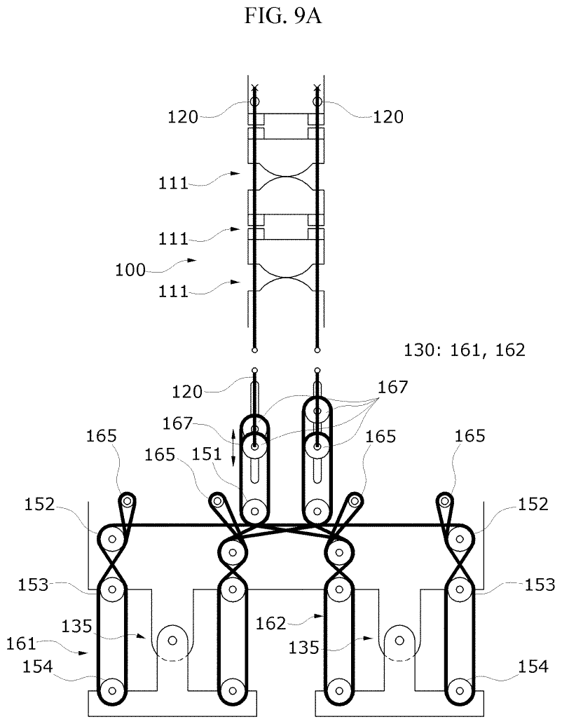

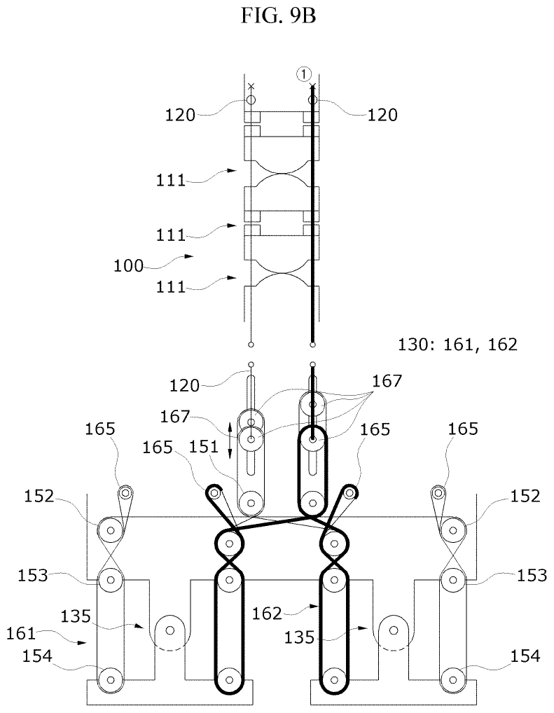

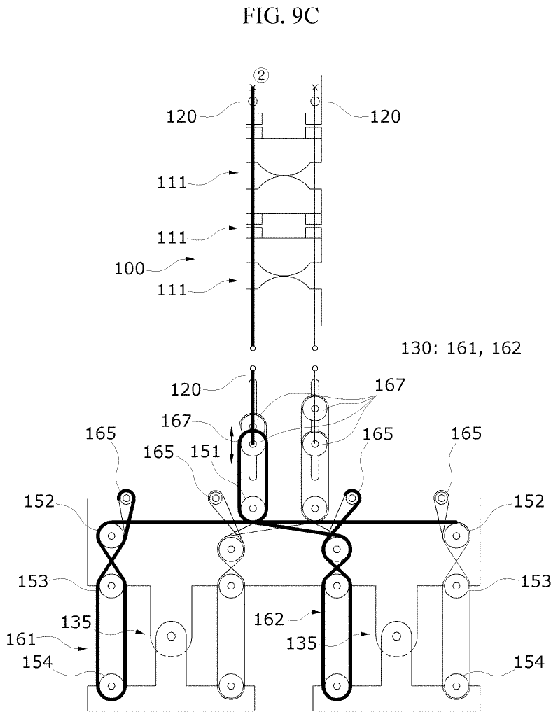

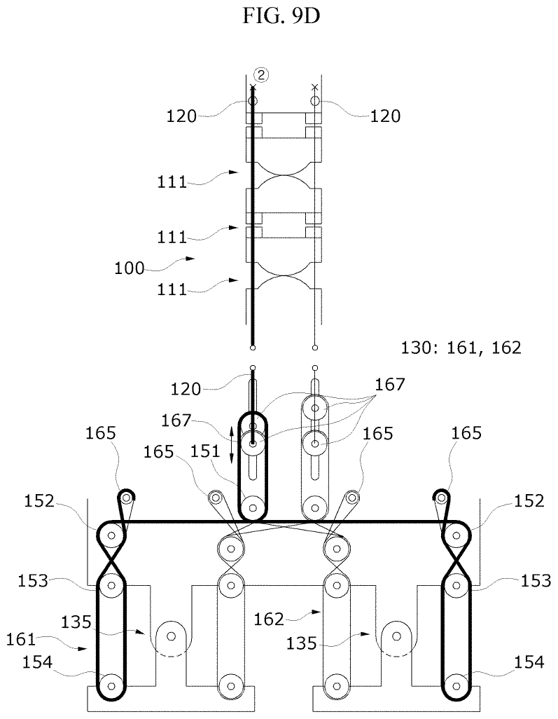

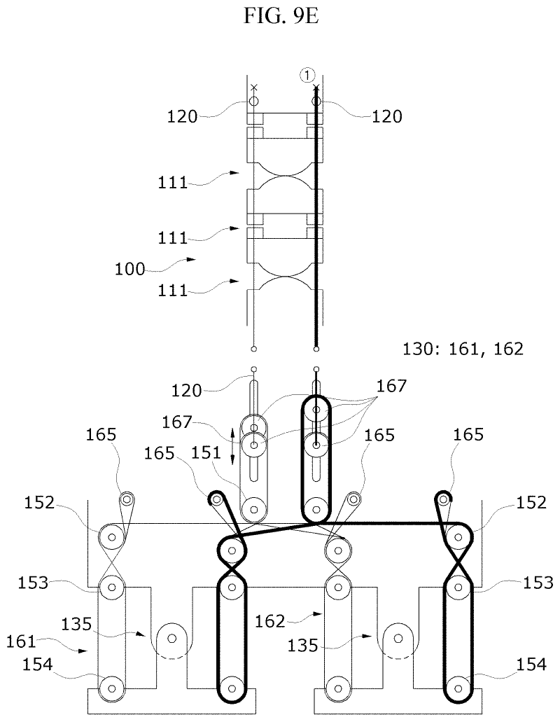

FIG. 9A is a conceptual diagram of modified cable driving in which sliding idlers which may perform translation movement are applied in an endoscope cable actuation mechanism according to yet another embodiment of the present disclosure. FIGS. 9B to 9E separately show the relation between wire cables 120 and various idlers for easy understanding thereof.

Referring to FIG. 9A and FIGS. 9B to 9E, the endoscope cable actuation mechanism according to the embodiment may further include sliding idlers 167 located to be close to the bending joint part 100 from the first idlers 151 and configured to adjust tension of the wire cables 120 while sliding according to the tension of the wire cables 120 because the wire cables 120 are disposed by surrounding the sliding idlers 167.

Fixing spools 165 are disposed in pairs to be located at both sides of the yaw driver 161 and the pitch driver 162, and the plurality of wire cables 120 forming different motions may be wound around and connected to the fixing spools 165.

As shown in FIG. 9A and FIGS. 9B to 9E, when the sliding idlers 167 which may perform translation are applied, minute cross-talk caused by movement of the wire cables 120 between the yawing operation and the pitching operation may be completely removed.

Meanwhile, in the above-described FIGS. 8A to 8E and FIGS. 9A to 9E, the cables which pass through the insides of the joints (numbers \textcircled{1} to \textcircled{4} disclosed in a cross-sectional view at an upper right portion in FIG. 8A) are a predetermined relation and should be assembled with a hinge joint of the driving part 130. For example, the \textcircled{1} and \textcircled{2} wire cables 120 which cause a plus(+) pitching operation and the \textcircled{3} and \textcircled{4} wire cables 120 which cause a minus(−) pitching operation should be located at a left side and a right side, respectively, at a hinge part of the pitch driver 162 in FIG. 8A. Alternatively, \textcircled{1} and \textcircled{2} wire cables 120 and \textcircled{3} and \textcircled{4} wire cables 120 should be located at the right side and the left side opposite to the above. Further, in the yawing operation, \textcircled{2} and \textcircled{3} wire cables 120 which cause a plus(+) yawing operation and \textcircled{1} and \textcircled{4} wire cables 120 which cause a minus(−) yawing operation should be oppositely located at a hinge part of the yaw driver 161 at a left side in FIG. 8A.

In addition, a design may depart from a planar conceptual structure so far and may be changed to a stereoscopic structure. The stereoscopic design may solve a problem in which occurrences of friction due to a difference between heights of the pulleys increase.

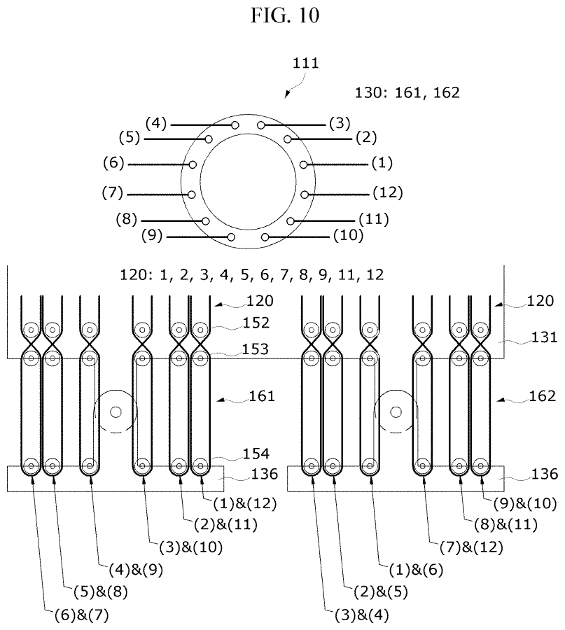

FIG. 10 is a layout diagram of twelve cables configured to drive the two-degree-of-freedom joint in an endoscope cable actuation mechanism according to yet another embodiment of the present disclosure.

Referring to FIG. 10, the wire cables 120 may be disposed at predetermined angle intervals along a circumferential direction and have freedom degrees of motion with respect to an axis of the bending joint part according to the number of the pairs of wire cables 120. FIG. 10 shows an example of locations of a plurality of idlers 140 through which the cables pass in the case in which the twelve cables are used to drive the two-degree-of-freedom joint. As shown in FIG. 10, a provided cable actuation mechanism has no limitation for the number of used cables. In FIG. 10, a combination of numbers (1) to (12) which are denoted to wire cables 120 refers to disposition for the idlers of the wire cables 120 disposed on a cylindrical cross-sectional surface above.

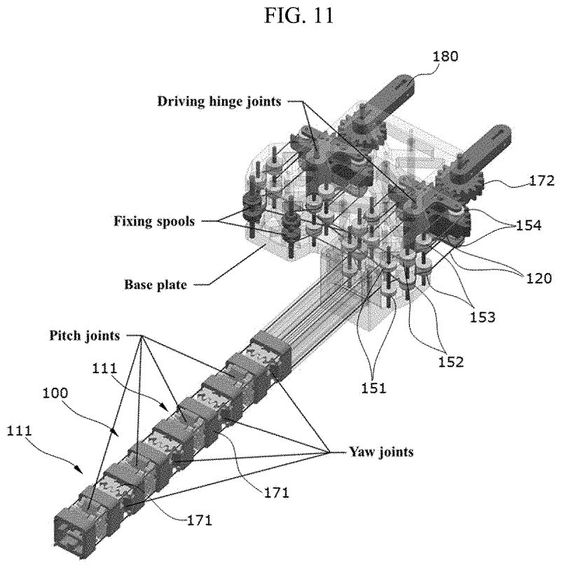

FIG. 11 is a three-dimensional (3D) detailed design view of the two-degree-of-freedom joint and a cable driving part configured to drive the two-degree-of-freedom joint in an endoscope cable actuation mechanism according to yet another embodiment of the present disclosure. FIG. 11 shows 3D design modeling to which the conceptual bending joint parts 100 and driving parts 130 of the embodiments are applied.

The two-degree-of-freedom bending joint part 100 includes eight units of pulleyless rolling joints 111, and four of the above are pitching joints and another four units are yawing joints. When a drivable angle of each of the unit joints is 45 degree, a maximum bending angle in yawing and pitching directions becomes 180 degree.

As shown, partial planetary gears 171 may be used to prevent sliding of the unit joints on a rolling surface. Further, two steering levers 180 may be applied to the driving part 130 without an actuator such as a motor or the like. The steering levers 180 coupled by two hinge joints of the driving part 130 and planetary gears 172 pull or release a driving cable due to rotation to cause the pitching operation and the yawing operation of the bending joint part 100 as in angulation knobs in a steerable endoscope. The components may be replaced by a motor to configure a robot system.

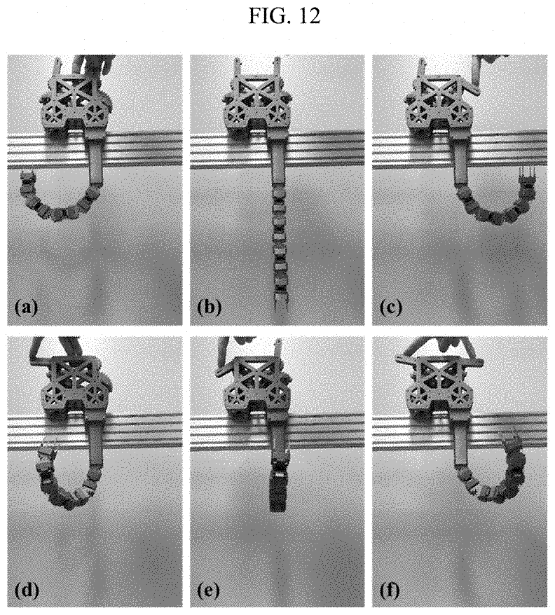

FIG. 12 is a joint bending state view in various directions using the steering levers in FIG. 11 in an endoscope cable actuation mechanism according to yet another embodiment of the present disclosure. FIG. 12 shows various position changes of the bending joint part 100 due to an operation of a provided cable driver.

Referring to FIG. 12, the left and right steering levers cause the pitching operation and the yawing operation ((a) to (c)), respectively. Joint bending ((d) to (0) in a diagonal direction may be performed by steering two steering levers at the same time, and tension of the driving cable may be maintained to be low in all positions.

Meanwhile, although the driving part is disclosed to have a lever manner in the embodiments, the driving part is not limited thereto, and a driving part including a joint, which has at least two-degree-of-freedom and is adjustable in frontward and backward directions and leftward and rightward directions, such as a universal joint, to drive pairs of wire cables at the same time, may be used. In a surgical instrument, since an operating instrument of a target corresponding to a driving part configured to adjust tension of wire cables while being operated by a user may use a universal joint or a spin joint generally applied to a laparoscopic endoscope surgical instrument, the joints may, of course, be selected for the driving parts in the embodiments.

An endoscope cable actuation mechanism according to the present disclosure can have a hinge joint structure which can form a mechanically complete match with a bending joint part at the end including a plurality of rolling joints, have a plurality of pulleys applied thereto for smooth operation of a cable, cause an occurrence of motion of one degree of freedom or two degrees of freedom or more according to a structure of the targeted bending joint part. Also, the endoscope cable actuation mechanism can be applicable to not only a conventional manual-type endoscope but also to a driving part of a surgical robot, and stably operate a miniaturized joint in which a small pulley therein is removed for miniaturization.

Further, a cable driving part in a new structure provided by the present disclosure can stably and smoothly drive a targeted joint using only low cable tension.

In addition, a mechanism provided by the present disclosure can replace a structure of the inside of a steering part such as a lever steered by a user in a conventional endoscope to prevent damage to a driving cable which frequently occurs, and can be further applied to a surgical tool to reduce the volume and manufacturing cost of the driving part.

The above description is only an exemplary description of the technical spirit of the present disclosure and may be variously changed and modified without departing from an essential characteristic of the present disclosure by those skilled in the art. Accordingly, the embodiments shown in the present disclosure are provided not to limit but to describe the technical spirit of the present disclosure, and the scope of the present disclosure is not limited by the embodiment. The scope of the present disclosure may be interpreted by the claims which will be described below, and the equivalents or all technical spirit in the equivalents may be included in the claims of the present disclosure.

What is claimed is:

1. An endoscope cable actuation mechanism comprising:
a bending joint part connected to a target;
a driving part configured to drive the bending joint part; and
at least one pair of wire cables configured to connect the bending joint part and the driving part,
wherein the bending joint part comprises at least one joint slected from a group of a pulleyless rolling joint, a pulleyless hinge joint, and a continuum joint, and
wherein the driving part comprises a driving main body; the pulleyless rolling joint or the pulleyless hinge joint coupled to the driving main body; and a driving bar connected to the pulleyless rolling joint or the pulleyless hinge joint of the driving main body and connected to the wire cables.

2. The endoscope cable actuation mechanism of claim 1, further comprising a plurality of idlers disposed at both sides of the driving main body so that the wire cables come into contact with the plurality of idlers and are bent, and configured to support and guide movement of the wire cables between the driving main body and the driving bar.

3. The endoscope cable actuation mechanism of claim 2, further comprising first sub idlers located at an edge of an inner wall of the driving main body and disposed in pairs at both sides of the driving main body so that the wire cables curvedly come into contact with and pass between the pair of first sub idlers.

4. The endoscope cable actuation mechanism of claim 3, further comprising second sub idlers located in pairs at both sides of the driving bar and configured to guide movement of the wire cables.

5. The endoscope cable actuation mechanism of claim 4, wherein multiple pairs of wire cables connect the bending joint part and the driving part for at least two degrees of freedom of the target.

6. The endoscope cable actuation mechanism of claim 5, wherein the bending joint part forms a yawing motion and a pitching motion with respect to the driving part by the multiple pairs of wire cables and forms a motion in which the yawing motion and the pitching motion are complexly applied.

7. The endoscope cable actuation mechanism of claim 6, wherein:
the at least one joint selected from a group of the pulleyless rolling joint, the pulleyless hinge joint, and the continuum joint is used as a joint for each of the yawing motion and the pitching motion, and the joints of the yawing motion and the pitching motion are alternately disposed in the bending joint part; and
the driving part comprises a yaw driver and a pitch driver for the yawing motion and the pitching motion, respectively.

8. An endoscope cable actuation mechanism comprising:
a bending joint part connected to a target;
a driving part configured to drive the bending joint part;
at least one pair of wire cables configured to connect the bending joint part and the driving part,
wherein the bending joint part comprises at least one joint slelected from a group of a pulleyless rolling joint, a pulleyless hinge joint, and a continuum joint,
wherein the driving part comprises a driving main body; the pulleyless rolling joint or the pulleyless hinge joint coupled to the driving main body; and a driving bar connected to the pulleyless rolling joint or the pulleyless hinge joint of the driving main body and directly or indirectly connected to the wire cables; and
a plurality of idlers disposed at both sides of each of the driving main body and the driving bar so that the wire cables come into contact with the plurality of idlers and are bent, and configured to support and guide movement of the wire cables between the driving main body and the driving bar,
wherein the plurality of idlers comprise first idlers located at both sides of the driving main body; second idlers located at both sides of the driving main body to be close to the first idlers; and third idlers located at both sides of the driving bar,
wherein each of the wire cables has one end portion which surrounds the first idler and is grounded and fixed to the driving main body and further comprises a wire loop cable having a loop shape and configured to surround and connect the second idler and the third idler.

9. The endoscope cable actuation mechanism of claim 8, wherein multiple pairs of wire cables connect the bending joint part and the driving part for at least two degrees of freedom of the target.

10. The endoscope cable actuation mechanism of claim 9, wherein the bending joint part forms a yawing motion and a pitching motion with respect to the driving part by the multiple pairs of wire cables and forms a motion in which the yawing motion and the pitching motion are complexly applied.

11. The endoscope cable actuation mechanism of claim 10, wherein:
the at least one joint selected from a group of the pulleyless rolling joint, the pulleyless hinge joint, and the continuum joint is used as a joint for each of the yawing motion and the pitching motion, and the joints of the yawing motion and the pitching motion are alternately disposed in the bending joint part; and
the driving part comprises a yaw driver and a pitch driver for the yawing motion and the pitching motion.

12. The endoscope cable actuation mechanism of claim 11, wherein:
the wire cables are disposed to connect the yaw driver and the pitch driver; and
the driving part has one end portion of the wire cable which is wound therearound and connected to be coupled to the driving main body, and further comprises a fixing spool to which the plurality of wire cables forming different motions are fixed.

13. An endoscope cable actuation mechanism comprising:
a bending joint part connected to a target;
a driving part configured to drive the bending joint part; and
at least one pair of wire cables configured to connect the bending joint part and the driving part,
wherein the bending joint part comprises at least one joint slelected from a group of a pulleyless rolling joint, a pulleyless hinge joint, and a continuum joint,
wherein the driving part comprises a driving main body; the pulleyless rolling joint or the pulleyless hinge joint coupled to the driving main body; and a driving bar connected to the pulleyless rolling joint or the pulleyless hinge joint of the driving main body and directly or indirectly connected to the wire cables; and
a plurality of idlers disposed at both sides of each of the driving main body and the driving bar so that the wire cables come into contact with the plurality of idlers and are bent, and configured to support and guide movement of the wire cables between the driving main body and the driving bar,
wherein the plurality of idlers comprise first idlers located at an inlet of the driving main body from the bending joint part; second idlers located to be spaced apart from the first idlers; third idlers located to be spaced apart from the second idlers; and fourth idlers located at the driving bar,
wherein the driving part comprises a yaw driver and a pitch driver for a yawing motion and a pitching motion, wherein each of the yaw driver and the pitch driver has the first to fourth idlers located at both sides thereof to form pairs, and the wire cables sequentially come into contact with the first idler, the second idler, the third idler, and the fourth idler and then with the third idler and the second idler of one of the yaw driver and the pitch driver, and sequentially come into contact with the first idler, the second idler, the third idler, and the fourth idler and then with the third idler and the second idler of the other one of the yaw driver and the pitch driver to have one ends fixed to the driving main body.

14. The endoscope cable actuation mechanism of claim 13, wherein multiple pairs of wire cables connect the bending joint part and the driving part for at least two degrees of freedom of the target.

15. The endoscope cable actuation mechanism of claim 14, wherein the bending joint part forms the yawing motion and the pitching motion with respect to the driving part by the multiple pairs of wire cables and forms a motion in which the yawing motion and the pitching motion are complexly applied.

16. The endoscope cable actuation mechanism of claim 15, wherein the at least one joint selected from a group of the pulleyless rolling joint, the pulleyless hinge joint, and the continuum joint is used as a joint for each of the yawing motion and the pitching motion, and the joints of the yawing motion and the pitching motion are alternately disposed.

17. The endoscope cable actuation mechanism of claim 13, wherein:
the wire cables are disposed to connect the yaw driver and the pitch driver; and
the driving part has one end portion of the wire cable which is wound therearound and connected to be coupled to the driving main body, and further comprises a fixing spool to which the plurality of wire cables forming different motions are fixed.

18. The endoscope cable actuation mechanism of claim 17, wherein:
fixing spools are located and disposed in pairs at both sides of each of the yaw driver and the pitch driver; and
a plurality of wire cables forming different motions are wound around and connected to the fixing spools.

19. The endoscope cable actuation mechanism of claim 14, wherein the wire cables are disposed at predetermined angle intervals along a circumferential direction and have degrees of freedom of motion with respect to an axis of the bending joint part according to the number of the pairs of wire cables.

20. The endoscope cable actuation mechanism of claim 13, wherein the driving part further comprises sliding idlers located to be close to the bending joint part from the first idlers and configured to adjust tension of the wire cables while sliding according to the tension of the wire cables as the wire cables are disposed so as to surround.
